(19) United States
(12) Patent Application Publication
(10) Pub. No.: US 2005/0240160 A1
(43) Pub. Date: Oct. 27, 2005

(54) Safety cap of syringe needle head

(76) Inventor: Chih Hsiung Lin, Taipei City (TW)

Correspondence Address: BUCKNAM AND ARCHER, 1077 NORTHERN BOULEVARD, ROSLYN, NY, 11576, US

(21) Appl. No.: 10/910,538

(22) Filed: Aug. 3, 2004

(30) Foreign Application Priority Data
Apr. 23, 2004 (TW) 93206263

(51) Int. Cl. A61M 5/00

(52) U.S. Cl. 604/263; 604/198

(57) ABSTRACT

A safety cap of syringe needle head comprises: a syringe body, having a medication chamber for the filling of medical liquid, one end of which can pressurize the medical liquid via a plunger, while the other end is formed with a mounting portion; a needle head, one end of which has an engagement portion being engaged with said mounting portion, the other end of which is fixedly attached with a needle via a seated portion for the releasing of medical liquid; a cap having an opened end and a closed end, and being capped on the outside of the needle head to protect the needle; wherein the opened end of the cap forms towards outside a funnel-shaped guiding portion with gradual enlargement, and the inner diameter of the opening end is tightly fitted with the outer diameter of the seated portion of the needle head. Accordingly, the needle is guided by the gradually enlarged funnel-shaped guiding surface so that the needle head can be easily inserted into the cap under safe condition.

### BACKGROUND OF THE INVENTION

(1) Field of the Invention

The present invention relates to a safe syringe, particularly to a safe cap for syringe needle head, wherein the needle head is guided by the gradually enlarged funnel-shaped guiding surface so that the needle head can be easily inserted into the cap under safe condition.

(2) Description of the Related Prior Art

The accidents, that medical personnel using a syringe for blood-drawing or medication injection are often pierced by careless handling of used needle of the syringe, even worse, infected with virus by this piercing from needle, have been uninterruptedly happened. Therefore, it is not an overstatement that medical personnel belong to a highly dangerous group of piercing accident by needle. In order to prevent needle from accidental piercing, a pulling device is provided at the front end of the plunger in the pushing direction, which can be engaged with the needle head after the medical liquid is pushed up, whereby the needle head is pulled and retracted into the syringe by the pulling back of the plunger in reverse direction. Then, all are disposed together. In ideal case, this type of syringe can attain safety effect to a certain degree. However, the components of this type of syringe are expensive due to their complicated structures, which result in high cost for a disposable type of syringe.

A conventional syringe is shown in FIGS. 9-11. As shown in FIG. 9, a conventional needle cap (10) is cylindrical in its shape and has an opened end (12) and a closed end (14) with the diameter of the opened end (12) being about the same as the diameter of the closed end (14). As shown in FIGS. 10 and 11, the inner diameter of the opened end (12) of the cap (10) is fitted with the outer diameter of the needle seated portion (18) of the syringe (16), therefore the bore diameter of the opened end (12) is made relatively small. When the needle seated portion (18) is intended to be inserted into the cap (10) after the injection of syringe (16) is finished, the needle head (19) is not easy to aim at the bore of the cap (10), so that the needle might slip to pierce the medical personnel's hand which holds the syringe (10), and cause the infection by virus. Thus, the safety of conventional syringe still has room for further improvement.

### SUMMARY OF THE INVENTION

In view of the defects in using conventional injector, the inventor of the present invention considers that injector should have high safety at lowest cost in ideal case. Therefore, the object of the invention is to provide a safety cap for syringe needle head with low cost.

Above and other objects will be attained by the safety cap for syringe needle head of present invention, comprising: a syringe body, having a medication chamber for the filling of medical liquid, one end of which can pressurize the medical liquid via a plunger, while the other end is formed with a injecting bore, and a mounting portion being formed at the injecting bore; a needle head, one end of which has an engagement portion being engaged with said mounting portion, the other end of which is fixedly attached with a needle via a seated portion for the releasing of medical liquid; a cap having an opened end and a closed end, and being capped on the outside of the needle head to protect the needle; wherein the opened end of the cap forms towards outside a funnel-shaped guiding portion with gradual enlargement, and the inner diameter of the opening end is tightly fitted with the outer diameter of the seated portion of the needle head.

According to this invention, few parts of the structure are changed in such a way that these changes do not procure cost-up. By the guiding of the funnel-shaped guiding surface, the needle head can be easily inserted into the cap under safe condition.

### A BRIEF DESCRIPTION OF THE DRAWINGS

FIG. 1 is a perspective view of the first embodiment of the safety cap of the present invention.

FIG. 2 is a sectional schematic view showing the combination of the safety cap with the syringe needle head of FIG. 1.

FIG. 3 is a schematic partly brokened view showing the safety cap and the concerned syringe of the present invention.

FIG. 4 is a schematic partly brokened view showing the safety cap and another concerned syringe of the present invention.

FIG. 5 is a sectional schematic view of the second embodiment of the safety cap of the present invention, wherein the cap is shown to be separated from the needle head.

FIG. 6 is a sectional schematic view showing the combination of the safety cap with the syringe needle head of FIG. 5.

FIG. 7 is a sectional schematic view of a derivative variation of the safety cap of the present invention.

FIG. 8 is a sectional schematic view of another derivative variation of the safety cap of the present invention.

FIG. 9 is a perspective schematic view showing a conventional syringe needle cap.

FIG. 10 is a side schematic view showing the combination of a syringe needle and a conventional cap.

FIG. 11 is a schematic view showing the syringe needle and a conventional cap in using status.

### DETAILED DESCRIPTION OF THE DRAWINGS

The technical features of the invention will be further understood by the following detailed description of a preferred embodiment. This preferred embodiment, merely for illustrative purpose, not to limit the scope of present invention, will become more apparent by the detailed description in conjunction with the following accompanied drawings.

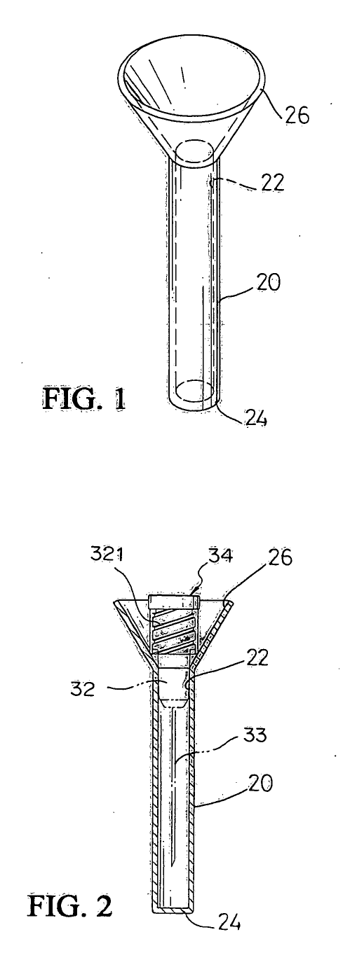

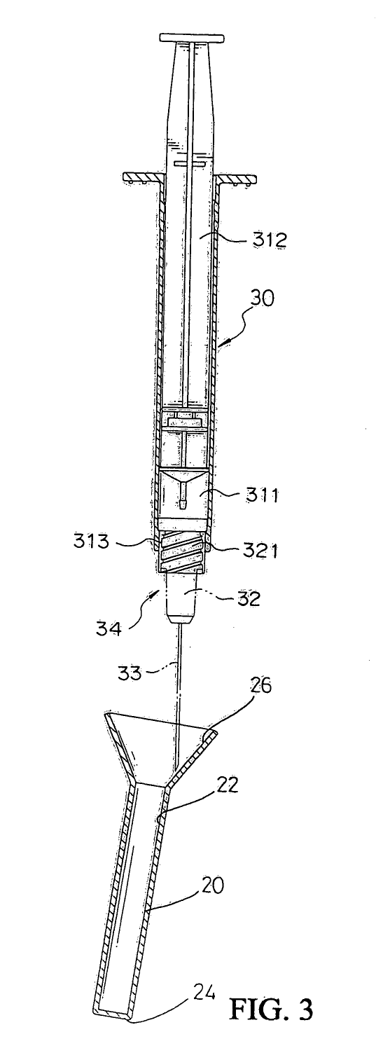

Firstly, referring to FIGS. 1, 2 and 3, a safety cap 20 of a needle head of the first embodiment of present invention is shown, while the concerned syringe has the conventional structure. The syringe body 30 has a medication chamber 311 for the filling of medical liquid, one end of which is provided with a plunger 312, the advancement of which in forward direction can pressurize the medical liquid, while the other end being formed with a injecting bore, and a mounting portion 313 being formed at the injecting bore; a needle head 34, one end of which has an engagement portion 321 being engaged with said mounting portion 313, the other end of which is fixedly attached with a needle 33 via a seated portion 32 for the releasing of medical liquid (as shown in FIG. 3); a cap 20 having an opened end 22 and a closed end 24 and being capped on the outside of the needle head 34 to protect the needle portion 33. The difference of the present invention from conventional structure is that the opened end 22 of the cap 20 forms towards outside a funnel-shaped guiding portion 26 having its bore size gradually enlarged, and the inner diameter of the opened end 22 is tightly fitted with the outer diameter of the seated portion 32 of the needle head 34(as shown in FIGS. 1 and 2).

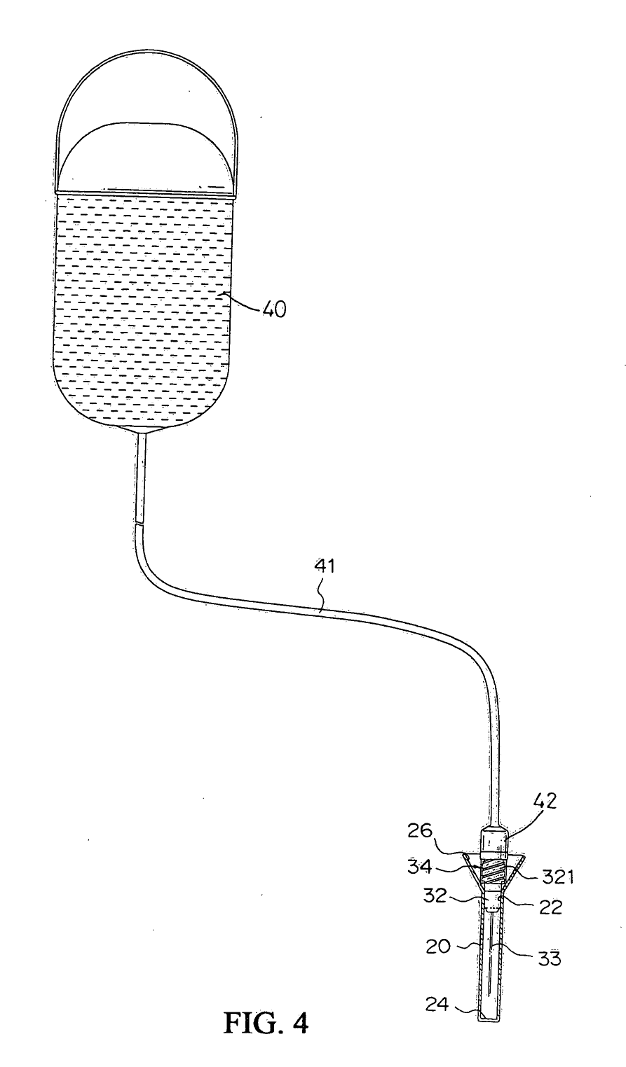

The safety cap 20 of needle head of the present invention as shown in FIG. 4 cooperates with another type of syringe, such as a drip-feed type of syringe etc., which comprises a chamber body 40 for the filling of medical liquid; a conduit 41, one end of which is connected on the chamber body 40 for the delivery of medical liquid, while the other end of which is formed with a mounting portion 42; a needle head 34, one end of which has an engagement portion 321 being engaged with said mounting portion 42, the other end of which is fixedly attached with a needle portion 33 via a seated portion 32 for the releasing of medical liquid; a cap 20 with an opened end 22 and a closed end 24 and being capped on the outside of the needle head 34 to protect the needle portion 33, and the opened end 22 of the cap 20 forms towards outside a funnel-shaped guiding portion 26 having its bore size being gradually increasing, and the inner diameter of the opened end 22 is tightly fitted with outer diameter of the seated portion 32 of the needle head 34.

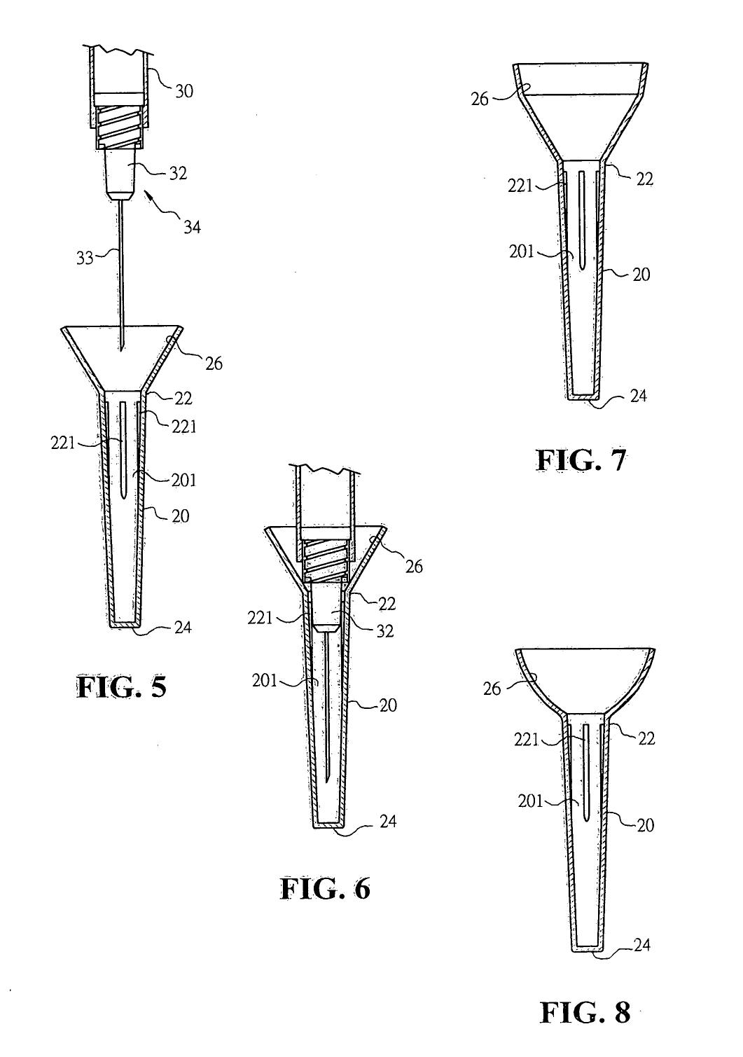

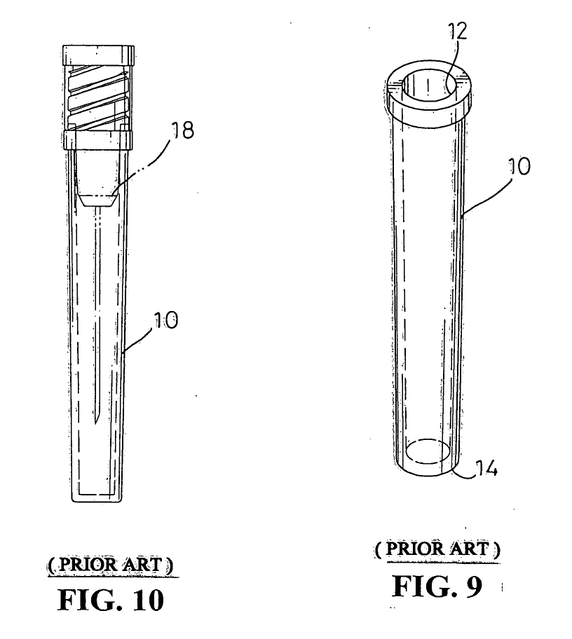

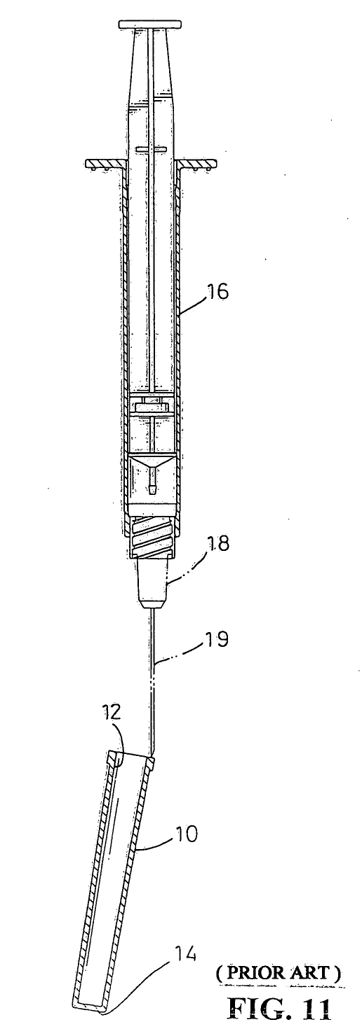

Referring to FIGS. 5 and 6, the safety cap 20 of the needle head of the second embodiment of present invention is shown. The difference of this embodiment with the previous embodiment is that the diameter of the bore 201 of the cap 20 is gradually decreased from the opened end towards the closed end, and that a plurality of ribs 221 extending in the axial direction are provided at equal interval along inner circumferential direction of the opened end 22. A space formed by the encircling of the ribs 221 is tightly fitted with the seated portion 32 of the needle head 34 (as shown in FIG. 6). In addition, examples of the variation of the present invention are shown in FIGS. 7 and 8, respectively. The guiding portion 26 of the cap 20 is formed of a funnel shape with its bore diameter decreasing from outside towards the opened end 22. The guiding portion 26 is allowed to have a radius of curvature, or is constructed by cone-shaped surface with any different cone angle. In these cases, it will become apparent to those who are skilled in this art that both structures will not depart from the object and effect of the present invention.

According to this invention, the guiding portion 26 in the above embodiment is formed with an enlarged receiving bore at the exterior of the cap 20 so that the cross sectional area of the receiving bore is several times of that of the opened end 22. This will allow the needle portion 33 of the needle head 34 to easily aim at the target bore during insertion. Furthermore, the guiding portion 26 is formed with a funnel shape, the bore diameter of which is decreasing from the outside towards the opened end 22. The needle 33 of the needle head 34 can be guided easily to the bore of the opened end 22 so that the needle head 34 can be easily inserted into the cap 20 under safe condition.

Based on the foregoing, the safety cap for syringe needle of this invention merely change partial structure of the conventional cap without causing any additional cost-up. The needle head can be guided by an enlarged funnel-shaped guiding surface so as to be easily inserted into the cap with high safety. No doubt, it is a novel, progressive invention having industrial availability.

The above-mentioned are preferred embodiments for illustrative purpose only and should not be consider as limitation on the scope of the present invention. It is evident that many alternatives, modifications and variations will be embraced within the scope and spirit of the present invention, which is clearly defined by the appended claims.

1. A safety cap of syringe needle head for use with a syringe, comprising:
a syringe body for the filling of medical liquid, one end of which has a pressurizing portion for pressurizing the medication liquid, while the other end is formed with a injecting bore and a mounting portion is formed at the injecting bore;
a needle head, one end of which has an engagement portion being engaged with said mounting portion;
a cap having an opened end and a closed end;
wherein the opened end of the cap forms towards outside a funnel-shaped guiding portion, and the inner diameter of the opening end is tightly fitted with the outer diameter of the seated portion of the needle head.

2. A safety cap of syringe needle head for use with a syringe, said syringe comprising:
a syringe body for the filling of medical liquid and delivering medical liquid via a conduit, a injecting bore being formed at the outlet of said conduit, and a mounting portion being formed at said injection bore;
a needle head, one end of which has an engagement portion being engaged with said mounting portion:
a cap having an opened end and a closed end:
wherein the opened end of the cap forms towards outside a funnel-shaped guiding portion, and the inner diameter of the opening end is tightly fitted with the outer diameter of the seated portion of the needle head.

3. A safety cap of syringe needle head as claimed in claim 1, wherein a plurality of ribs extending in axial direction are provided at equal interval along the inner circumferential direction of said opened end of said cap.

4. A safety cap of syringe needle head as claimed in claim 3, wherein a space formed by the encircling of said plurality of ribs is fitted with said seated portion of the needle head.

5. A safety cap of syringe needle head as claimed in claim 1, wherein the inner diameter of said opened end is gradually decreased towards the closed end.

6. A safety cap of syringe needle head as claimed in claim 1, wherein said cap guiding portion is formed with a funnel shape, the bore of which is gradually decreased from outside towards the opened end.

7. A safety cap of syringe needle head as claimed in claim 6, wherein said guiding portion has a radius of curvature.

8. A safety cap of syringe needle head as claimed in claim 6, wherein said guiding portion has a cone-shaped surface with any different cone angle.

9. A safety cap of syringe needle head as claimed in claim 2, wherein a plurality of ribs extending in axial direction are provided at equal interval along the inner circumferential direction of said opened end of said cap.

10. A safety cap of syringe needle head as claimed in claim 9, wherein a space formed by the encircling of said plurality of ribs is fitted with said seated portion of the needle head.

11. A safety cap of syringe needle head as claimed in claim 2, wherein the inner diameter of said opened end is gradually decreased towards the closed end.

12. A safety cap of syringe needle head as claimed in claim 2, wherein said cap guiding portion is formed with a funnel shape, the bore of which is gradually decreased from outside towards the opened end.

13. A safety cap of syringe needle head as claimed in claim 12, wherein said guiding portion has a radius of curvature.

14. A safety cap of syringe needle head as claimed in claim 13, wherein said guiding portion has a cone-shaped surface with any different cone angle.
